(19) United States
(12) Patent Application Publication
(10) Pub. No.: US 2006/0121163 A1
(43) Pub. Date: Jun. 8, 2006

(54) Beverage management system

(75) Inventor: Mark Holloway, Mosman Park (AU)

Correspondence Address: NIXON & VANDERHYE, PC, 901 NORTH GLEBE ROAD, 11TH FLOOR, ARLINGTON, VA, 22203, US

(73) Assignee: RXM Red Pty. Ltd., Western Australia (AU)

(21) Appl. No.: 11/293,225

(22) Filed: Dec. 5, 2005

(30) Foreign Application Priority Data
Dec. 3, 2004 (AU) 2004906912

(51) Int. Cl. G01N 33/02

(52) U.S. Cl. 426/231

(57) ABSTRACT

A beverage measurement system for a beverage container such as wine bottle includes a series of markings 12 provided on an exterior surface of the wine bottle 14 and precisely positioned to indicate, from the level of beverage remaining in the container, when a measured quantity of the beverage has been dispensed from the container. The measured quantity relates to a particular material content of the beverage in the bottle 14 such as alcohol content, so that a consumer can determine how much alcohol has been consumed. The alcohol content may be represented as the number of “standard drinks”. A drinking vessel is also described.

### BACKGROUND OF THE INVENTION

This application claims the benefit of Australian provisional patent application 2004906912 filed on Dec. 3, 2004, the entirety of which is incorporated by reference.

The present invention relates to a beverage measurement system for indicating when a measured quantity of a beverage has been dispensed from a beverage container and relates particularly, though not exclusively, to such a system for indicating when a “standard drink” of an alcoholic beverage has been dispensed. A drinking vessel is also described.

Wine is the most widely consumed ‘drug’ in the world. The therapeutic benefits of wine consumption have been scientifically determined to lie within a fairly narrow range. The limits of this range vary with gender, age, pre-existing risk factors and diseases. Most epidemiological studies have confirmed that an average weekly consumption of 50-100 g of alcohol can reduce cardiovascular mortality by 25-30%. Other potential benefits relate to possible reduction in ischaemic stroke, macular degeneration and Alzheimer's disease. On the other hand, many studies have also shown that moderate to heavy regular drinking and binge drinking raises mortality to an even greater degree. Younger persons, those operating dangerous machines, pregnant women and other smaller groups are at serious risk. Drink-driving also continues to be a major contributor to road fatalities, killing drivers, passengers and pedestrians. Therefore it is in the interests of all that ways be found of assisting people to regulate their alcohol consumption.

Many countries have adopted a ‘standard drink’ guide to inform the consumer about ‘safe’ drinking levels. The Australian and European standard is based on 10 g (12.5 mls) of alcohol per standard drink. In the UK and Ireland a standard drink contains 8 g of alcohol and in the US and Canada it is 14 g of alcohol per standard drink. However studies had shown that most people have no idea how to measure a standard drink. Current wine labels are required to provide information regarding the alcohol content (%) and the number of standard drinks contained in the bottle. The tolerance for error in this labelling information can be up to 20%. A standard bottle of wine in Australia contains 750 ml of wine with an alcohol content of 12.0-14.5%, which equates to between 8-11 standard drinks. Notwithstanding the provision of this information, most people overestimate a standard drink by up to 50%. Furthermore conventional wine glasses can typically hold up to two standard drinks.

The present invention was developed with a view to providing a beverage measurement system for indicating when a “standard drink” of wine has been dispensed from a wine bottle. However, it will be evident that the same system can also be used for indicating when a measured quantity of a beverage has been dispensed from a beverage container, wherein the measured quantity relates to some other particular material content of the beverage, such as calorie content or carbohydrate content, and so on.

References to prior art in this specification are provided for illustrative purposes only and are not to be taken as an admission that such prior art is part of the common general knowledge in Australia or elsewhere.

### BRIEF DESCRIPTION OF THE INVENTION

A beverage measurement system for a beverage container, the system comprising:

a series of markings provided on an exterior surface of the container and precisely positioned to indicate, from the level of beverage remaining in the container, when a measured quantity of the beverage has been dispensed from the container, and wherein said measured quantity relates to a particular material content of the beverage in the container, so that a consumer can determine how much of said material content has been consumed.

Preferably said markings are printed on a label attached to the exterior surface of the container. Typically said label is elongate in shape and is attached to the exterior surface longitudinally of the bottle. Alternatively, said markings are embossed into the exterior surface of the container.

Typically said beverage container is a wine bottle, said beverage is wine, and said particular material content is the alcohol content of the wine. Advantageously said measured quantity is a ‘standard drink’, representing a standard measure quantity of alcohol according to the national standards of the country concerned. The Australian and European standard is based on 10 g (12.5 mls) of alcohol per standard drink. Preferably said label has markings at spaced intervals indicating from top to bottom, in ascending order, the number of standard drinks that have been dispensed from the bottle.

According to a further aspect of the disclosed beverage measurement system there is provided an accessory in the form of a drinking vessel suitable for use with the beverage measurement system for the beverage container, the drinking vessel being provided with a series of markings on the external surface thereof, the markings being precisely positioned to indicate, from the level of the liquid in the vessel, when a measured quantity has been dispensed into the vessel, and wherein said measured quantity relates to a particular material content of the liquid in the vessel, so that a consumer may determine the quantity of said material content that is to be consumed.

The drinking vessel is preferably a wine glass and the liquid is preferably wine. The material content of the liquid is preferably the alcohol content of the wine. Typically the markings are etched into the exterior surface of the vessel. The markings are conveniently positioned circumferentially around the exterior surface of the wine glass.

Advantageously said measured quantity is a ‘standard drink’, representing a standard measure quantity of alcohol according to the national standards of the country concerned. The Australian and European standard is based on 10 g (12.5 mls) of alcohol per standard drink.

Preferably the markings on the drinking vessel are at spaced intervals indicating from bottom to top, in ascending order, the number of standard drinks that have been poured into the vessel.

The disclosed beverage measurement system also provides a drinking vessel with a series of markings on the external surface thereof, the markings being precisely positioned to indicate, from the level of the liquid in the vessel, when a measured quantity has been dispensed into the vessel, and wherein said measured quantity relates to a particular material content of the liquid in the vessel, so that a consumer may determine the quantity of said material content that is to be consumed.

The drinking vessel is preferably a wine glass and the liquid is preferably wine. The material content is preferably the alcohol content of the wine. Typically the markings are etched into the exterior surface of the vessel. The markings are conveniently positioned circumferentially around the exterior surface of the wine glass.

Advantageously said measured quantity is a ‘standard drink’, representing a standard measure quantity of alcohol according to the national standards of the country concerned. The Australian and European standard is based on 10 g (12.5 mls) of alcohol per standard drink.

Preferably the markings on the drinking vessel are at spaced intervals indicating from bottom to top, in ascending order, the number of standard drinks that have been poured into the vessel.

Throughout the specification, unless the context requires otherwise, the word “comprise” or variations such as “comprises” or “comprising”, will be understood to imply the inclusion of a stated integer or group of integers but not the exclusion of any other integer or group of integers.

### BRIEF DESCRIPTION OF THE DRAWINGS

The nature of the invention will be better understood from the following detailed description of a preferred embodiment of the beverage measurement system, given by way of example only, with reference to the accompanying drawings, in which:

FIG. 1 shows a typical front label for a wine bottle;

FIG. 2 shows a preferred embodiment of a measurement label employed in the beverage measurement system of the present invention;

FIG. 3 shows a front view of a wine bottle with the label of FIG. 1 attached;

FIG. 4 shows a side view of the wine bottle of FIG. 3 with the label of FIG. 2 attached;

FIG. 5 shows a perspective view of the wine bottle of FIG. 3 with a level of wine indicated in dotted lines; and

FIG. 6 shows a perspective view of a wine glass according to an embodiment of the invention.

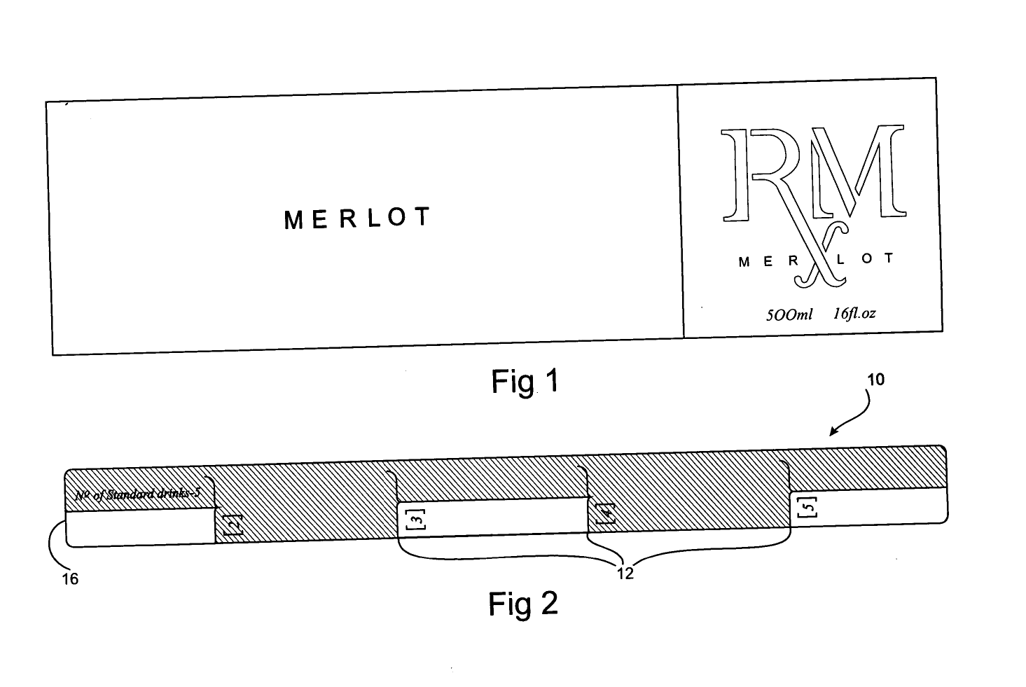

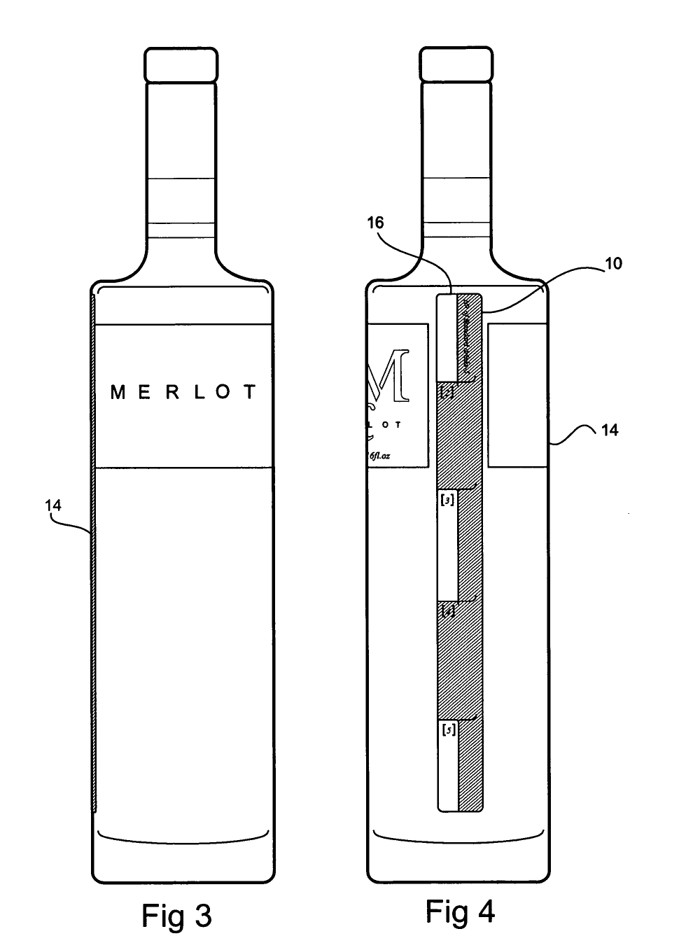

### DETAILED DESCRIPTION OF THE INVENTION

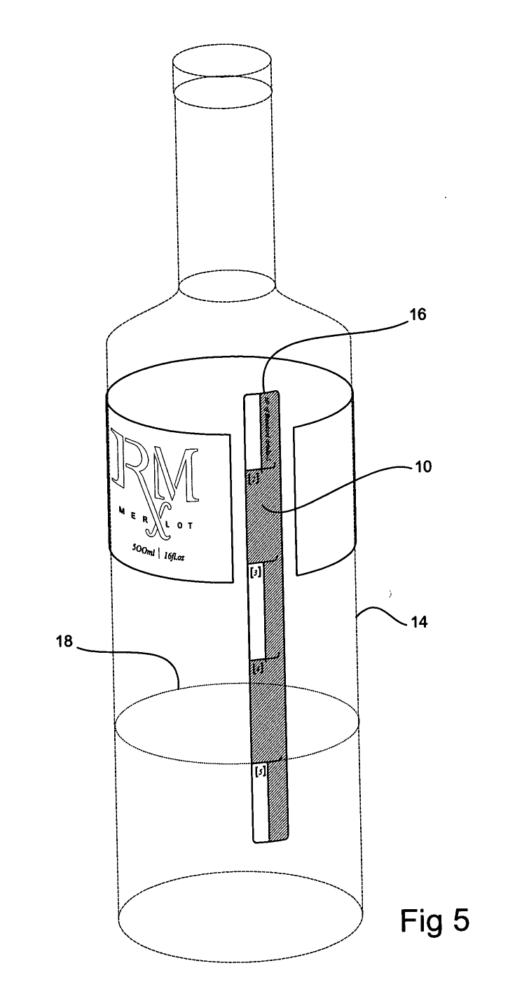

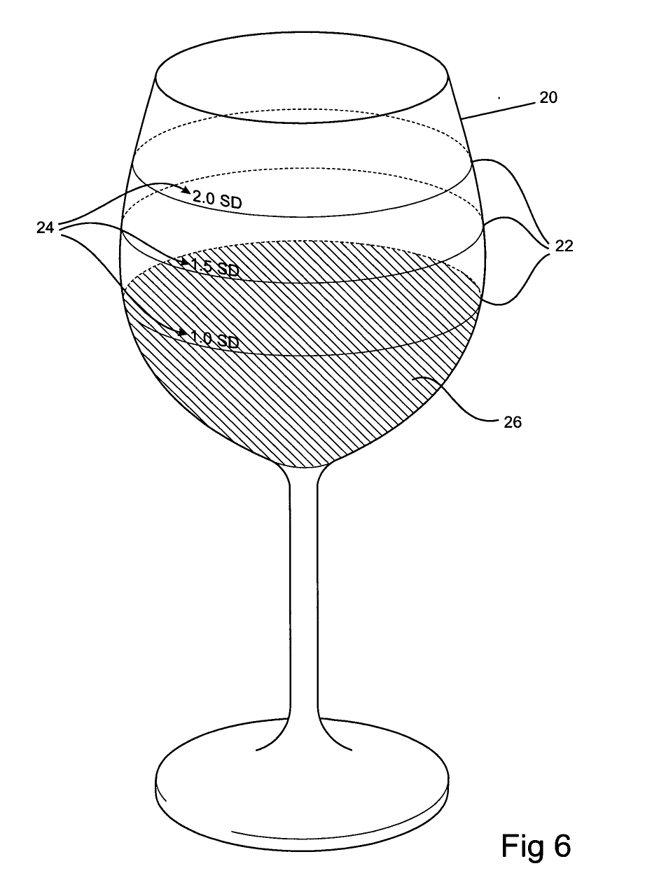

A preferred embodiment of the beverage measurement system is illustrated in FIGS. 2, 4 and 5 and comprises an elongate label 10 in the form of a strip, on which a plurality of markings 12 have been printed at spaced intervals. The label 10 is designed to be attached vertically to a wine bottle 14 as shown in FIG. 4, to provide an indication to a consumer of the number of “standard drinks” that have been dispensed from the bottle. As noted above, in Australia and Europe, a “standard drink” is equivalent to 10 g (12.5 mls) of alcohol. However the actual volume of wine in a standard drink will depend on the alcohol content of the wine. Thus, if we assume the alcohol content of the wine to be 12.5%, then the volume of wine in a standard drink will be 100 mls. In the illustrated embodiment, therefore, the spaced intervals between the markings 12 each represent a volume of 100 ml of wine. However the actual spacing between the markings 12 may vary depending on the internal shape of the wine bottle 14.

In the illustrated embodiment a 500 ml “Kendo” wine bottle has been used, and therefore the bottle 14 should hold exactly five (5) “standard drinks” of wine with an alcohol content of 12.5%. The internal shape of a Kendo wine bottle is tapered although the external shape and size is similar to that of a conventional 750 ml wine bottle. Hence the markings 12 on the label 10 are not at equidistant intervals, since the internal cross-sectional area of the bottle, (and hence the volume), varies vertically. The position of each of the markings 12 on the label 10 is therefore precisely calculated to provide a clear demarcation of the meniscus level of wine into five standard drinks. The markings 12 indicate from top to bottom, in ascending order, the number of standard drinks that have been dispensed from the bottle. Advantageously the label 10 is printed with alternating colors or shades of color between the markings 12 to aid in visually distinguishing the markings 12. Hence, as the consumer pours the wine, he/she will be able to ascertain, from the level of wine remaining in the bottle, how much alcohol has been dispensed into their glass and how much alcohol has been consumed. Thus, for example, when the meniscus of the wine remaining in the bottle reaches the top edge 16 of the label 10, then the consumer knows that one (1) standard drink has been poured. When the meniscus reaches the first marking 12 below the top edge 16 of the label 10, then the consumer knows that two (2) standard drinks have been consumed.

Advantageously the wine bottle 14 is also provided with a unique neck tag (not shown) on which precise information is given for those at risk when consuming alcohol. This information may include recommended healthy doses for particular individuals based on age, gender and other pre-existing medical factors; safe levels for acute and long term consumption; and further information to allow individuals to maximize the health benefits of wine alcohol consumption and minimize the potential harm alcohol consumption.

The beverage measurement system and the wine bottle 14 may also be used in association with a wine glass 20 as shown in FIG. 6. The wine glass 20 has a plurality of markings in the form of rings 22 etched into the exterior surface of the glass 20. The rings 22 extend around the circumference of the glass 20 for ease of reference all the way round the glass. The rings 22 are accurately positioned to indicate when a measured quantity of wine (according to alcohol content) has been poured into the glass 20. The alcohol content is given in the form of “standard drink” size, for example according to Australian standards, 10 g alcohol corresponds to 1 standard drink, 15 g alcohol corresponds to 1.5 standard drinks, and 20 g alcohol corresponds to 2 standard drinks, and so on.

As shown in FIG. 6, the standard drink (SD) sizes 24 are printed on the glass to denote the number of standard drinks corresponding to the amount of alcohol in the wine 26 in the glass 20. For example, in FIG. 6 the amount of wine 26 in the glass 20 corresponds to one standard drink (or 10 g of alcohol). Clearly, the amount of alcohol could be indicated against the markings 22 instead of representing these amounts by “standard drink” size.

Thus the wine glass 20 provides the consumer with a measure of the amount of alcohol (by standard drink size) in the glass they are about to consume. In this way, the uncertainty and confusion surrounding the alcohol content of the glass of wine is avoided. This is particularly important in light of the many varying glass sizes and drink sizes for wines and alcoholic beverages that are offered to consumers presenting a health and safety risk.

Many persons wish to ensure that they consume safe amounts of alcohol. It is well accepted by government and health authorities that the potential health benefits of alcohol consumption are limited to small regular quantities. However, to date there has been no convenient and reliable way of monitoring alcohol consumption. The beverage measurement system and the drinking vessel of the invention provide a means for consumers to monitor the amount of alcohol they consume in an accurate way.

Clearly the beverage measurement system and the drinking vessel of the invention may be adapted as appropriate according to the various “standard drink” requirements of each particular country. For example, whilst Australian and European standards set the standard drink level at 10 g alcohol per standard drink, the US and Canada have a standard drink level of 14 g per standard drink. The series of markings on the bottle label or on the wine glass may be adjusted accordingly.

Recently, there has been steadily increasing market for so-called ‘sports drinks’ which are specially formulated beverages designed to replace necessary minerals and salts lost during physical exertion, and to replenish energy consumed during heavy exercise. Other formulations are intended to assist with weight-loss, body building or reduction in body mass, etc. Ideally, the consumption of such specially formulated beverages should also be regulated before, during and/or after physical exercise in order to maximize the potential benefits. Therefore the beverage system of the present invention may also find application in the field of sports drinks, so that consumers can accurately monitor their calorie, carbohydrate, mineral, protein, etc intake.

Although in the preferred embodiment of the beverage measurement system the series of markings are printed on a label which is carefully positioned and attached to an exterior surface of the container, it is also possible to provide the markings directly on the exterior surface of the container. This may be done either by printing the markings directly onto the exterior surface of the container, or by embossing the markings into the exterior surface of the container. This has the advantage that a separate label does not have to be attached to the bottle and the markings can be included at the point of manufacture of the bottles. However, this option has the disadvantage that only wine with a particular alcohol content can be contained in these bottles.

Now that a preferred embodiment of the beverage measurement system has been described in detail, it will be apparent that it has a number of advantages, including the following:

(i) It permits consumers of alcoholic beverages to accurately monitor their alcohol consumption, with the attendant health and safety benefits;

(ii) It allows consumers to readily determine how many ‘standard drinks’ they have consumed, the ‘standard drink’ measure being a concept with which consumers are very familiar;

(iii) It is inexpensive to manufacture and apply to beverage containers; and

(iv) It can be readily modified to suit a wide range of beverages in various shapes and sizes of containers.

It will be readily apparent to persons skilled in the relevant arts that various modifications and improvements may be made to the foregoing embodiments, in addition to those already described, without departing from the basic inventive concepts of the present invention. For example, the measurement label of the preferred embodiment may be combined with a standard wine label to form a single integral label, which is then attached to the exterior surface of the bottle. Therefore, it will be appreciated that the scope of the invention is not limited to the specific embodiments described.

What is claimed is:

1. A beverage measurement system for a beverage container, the system comprising:
a series of markings provided on an exterior surface of the container and precisely positioned to indicate, from the level of beverage remaining in the container, when a measured quantity of the beverage has been dispensed from the container, and wherein said measured quantity relates to a particular material content of the beverage in the container, so that a consumer can determine how much of said material content has been consumed.

2. A beverage measurement system according to claim 1 wherein the markings are printed on a label attached to the exterior surface of the container.

3. A beverage measurement system according to claim 2 wherein the label is elongate in shape and is attached to the exterior surface longitudinally of the bottle.

4. A beverage measurement system according to claim 2 wherein the label has markings at spaced intervals indicating from top to bottom, in ascending order, the number of standard drinks that have been dispensed from the bottle.

5. A beverage measurement system according to claim 1 wherein said markings are embossed into the exterior surface of the container.

6. A beverage measurement system according to claim 1 wherein the beverage container is a wine bottle, and the beverage is wine.

7. A beverage measurement system according to claim 6 wherein the material content is the alcohol content of the wine.

8. A beverage measurement system according to claim 7 wherein the measured quantity is a ‘standard drink’, representing a standard measure quantity of alcohol according to the national standards of the country concerned.

9. A beverage measurement system according to claim 8 wherein a standard drink standard is equivalent to 10 g or 12.5 ml of alcohol.

10. A beverage measurement system according to claim 1, further comprising an accessory in the form of a drinking vessel suitable for use with the beverage measurement system for the beverage container, the drinking vessel being provided with a series of markings on the external surface thereof, the markings being precisely positioned to indicate, from the level of the liquid in the vessel, when a measured quantity has been dispensed into the vessel, and wherein said measured quantity relates to a particular material content of the liquid in the vessel, so that a consumer may determine the quantity of said material content of the liquid that is to be consumed.

11. A beverage measurement system according to claim 10 wherein the drinking vessel is a wine glass and the liquid is wine.

12. A beverage measurement system according to claim 11 wherein the material content of the liquid is the alcohol content of the wine.

13. A beverage measurement system according to claim 10 wherein the markings are etched into the exterior surface of the vessel.

14. A beverage measurement system according to claim 11 wherein the markings are positioned circumferentially around the exterior surface of the wine glass.

15. A beverage measurement system according to claim 12 wherein said measured quantity is a ‘standard drink’, representing a standard measure quantity of alcohol according to the national standards of the country concerned.

16. A beverage measurement system according to claim 15 wherein the markings on the drinking vessel are at spaced intervals indicating from bottom to top, in ascending order, the number of standard drinks that have been poured into the vessel.

17. A drinking vessel with a series of markings on the external surface thereof, the markings being precisely positioned to indicate, from the level of the liquid in the vessel, when a measured quantity has been dispensed into the vessel, and wherein said measured quantity relates to alcohol content of the liquid in the vessel, so that a consumer may determine the quantity of said alcohol that is to be consumed.

18. A drinking vessel according to claim 17 wherein the vessel is a wine glass and the liquid is wine.

19. A drinking vessel according to claim 17 wherein said measured quantity is a ‘standard drink’, representing a standard measure quantity of alcohol according to the national standards of the country concerned.

20. A drinking vessel according to claim 19 wherein the markings on the drinking vessel are at spaced intervals indicating from bottom to top, in ascending order, the number of standard drinks that have been poured into the vessel.
